(19) United States
(12) Patent Application Publication
(10) Pub. No.: US 2017/0224600 A1
(43) Pub. Date: Aug. 10, 2017

(54) Synergistic Compositions That Reduce UV-Induced Lipid Peroxidation, Formulations and Related Methods

(71) Applicant: Inolex Investment Corporation, Wilmington, DE (US)

(72) Inventors: Rocco Burgo, Mullica Hill, NJ (US); Daniel Winn, Kingston, NJ (US)

(21) Appl. No.: 15/427,700

(22) Filed: Feb. 8, 2017

(30) Foreign Application Priority Data
Feb. 10, 2016 (JP) 2016-023527

(51) Int. Cl. A61K 8/42; A61Q 19/08; A61Q 17/04; A61K 8/34; A61K 8/31; A61K 8/67; A61Q 19/00

(52) U.S. Cl. CPC A61K 8/42; A61K 8/676; A61Q 19/08; A61Q 19/00; A61K 8/678; A61K 8/347; A61K 8/31; A61Q 17/04; A61K 2800/42

(57) ABSTRACT

Disclosed is a composition of a synergistically effective ratio of an hydroxamic acid, salt and/or a complex thereof and an antioxidant. The composition is effective to reduce or prevent lipid peroxidation upon exposure of a lipid to UV light. Personal care and other formulations are included within the scope of the invention, as are the related methods of increasing the antioxidant potential of an antioxidant, of reducing a rate of peroxidation of constituent lipids in a personal care formulation over a time period and of reducing peroxidation of lipids within mammalian skin cells.

### BACKGROUND OF THE INVENTION

Solar radiation is a form of energy emitted by the sun and comprised of at least three spectra of electromagnetic waves: visible light, infrared radiation and ultraviolet radiation. Visible light is the portion of the spectrum represented by wavelengths of 400 to 700 nanometers. Infrared radiation is invisible radiant of wavelength of greater than about 700 nanometers (i.e., wavelength extending from the nominal red edged of the visible spectrum), which can be physically perceived as heat. Ultraviolet radiation has wavelengths in the range of about 400 nm to about 10 nm. Excessive exposure to solar radiation might result in harmful outcomes for living beings, including acute and immediate damage such as sunburn and erythema, and/or longer term damage, such as premature aging of skin and skin cancer. Exposure to UV radiation may be of particular seriousness, since it cannot be perceived by an individual so he or she cannot gauge his or her exposure level.

Ultraviolet radiation (UV) is further subdivided into UVA radiation (400 to 320 nm), UVB radiation (320 to 290 nm) and UVC radiation (290 to 10 nm). In general, UVC radiation is almost completely absorbed by the ozone layer and does not affect humans or other animals. However, UVB and UVA radiation are not completely absorbed by the atmosphere, and may make contact with and damage or degrade skin and other materials. Skin exposure to UVB radiation may cause immediate skin tanning and acute sunburn. Exposure to UVA radiation promotes melanin synthesis and delayed tanning, as well as oxidative photobiological reactions which may produce free radicals within the skin cells.

Both UVA and UVB are understood to have carcinogenic potential, though UVA is more closely associated with damage to dermal tissue (e.g., collagen degradation) and subsequent signs of premature aging. More specifically, UV radiation can cause damages to nucleic acids, proteins and lipids in skin cells. For instance, Punnonen, Puntala and Ahotupa (1991) report that both UVA and UVB radiation are capable of inducing lipid peroxidation reactions and inhibiting cellular antioxidant enzymatic systems in human keratinocytes (Punnonen, K., A. Puntala, and M. Ahotupa, “Effects of ultraviolet A and B irradiation on lipid peroxidation and activity of the antioxidant enzymes in keratinocytes in culture”, Photodermatol., Photoimmunol. & Photomed., 8, pp. 3-6 (1991)).

Further, Vile and Tyrrell (1995) observed that human skin fibroblast exposed to UVA radiation undergo deleterious lipid peroxidation promoted by UVA in reactions that also involve iron, singlet oxygen and hydrogen peroxide (Vile, G. F., and R. M. Tyrrell, “UVA radiation-induced oxidative damage to lipids and proteins in vitro and in human skin fibroblasts is dependent on iron and singlet oxygen”, Free Rad. Biol. Med., 18, pp. 721-730 (1995)).

Complementarily, Yin, Xu and Porter (2011) report that UV radiation excites intracellular oxygen-generating free radicals, also known as Reactive Oxygen Species (ROS), which eventually will interact with lipids, proteins and other structures, impairing normal cellular function (Yin, H., L. Xu, and N. A. Porter, “Free Radical Lipid Peroxidation: Mechanisms and Analysis”, Chem. Rev., 111, pp. 5944-5972 (2011)).

A “radical” or a “free radical” is a molecule with one or more unpaired electrons in its outer orbital shell. Free radicals are highly chemically reactive, often reacting with other molecules and forming new free radical species in a self-propagating chain reaction. Free radical damage to cell membrane lipids may cause the damaged membranes to lose their ability to transport oxygen, nutrients or water into cells. Such type of cell dysfunction is commonly associated with various pathological conditions and aging. Indeed, KR Patent 20010010240 mentions the use of triterpene derivatives as lipid peroxidation inhibitors, further characterizing then as performing as anti-cancer drugs and aging prevention agents.

In addition to the negative consequences peroxidation, particularly lipid peroxidation can have on the look and health of the skin, lipid peroxidation may affect the shelf stability of personal care and pharmaceutical products containing ingredients such as vegetable oils, vegetable waxes, butters, triglycerides and other unsaturated fatty materials. For example, in JP Patent H05320048, Shiseido Co. Ltd. provides a synergistic combination of δ-tocopherol and natural sphingosin to be used as a preservative in cosmetic formulations subject to lipid peroxidation to promote stability of the formulation.

Prior attempts to inhibit or prevent lipid peroxidation both in personal care/pharmaceutical products and living skin cells have been made. Sunscreen agents topically applied to skin serve to filter/absorb UV radiation, preventing ROS formation. Such agents may include octocrylene, octyl methoxycinnamate, benzophenone-3, benzophenone-4, octyl salicylate, butyl methoxydibenzoylmethan. However, the activity of these agents is dependent of dose, exposure wavelength and time. Moreover, many of these conventional sunscreen agents are poorly photostable and therefore do not provide longer lasting protection.

Antioxidant agents, such as ascorbic acid, sodium ascorbyl phosphate, ascorbyl palmitate, α-tocopherol, tocopheryl acetate, abutylated hydroxytoluene, butylated hydroxyanisole, phenolic compounds, carotenoids, and organic selenium, are capable of stabilizing free radicals, therefore interrupting chain reaction. Their activity is also dose-dependent and some are sensitive to light, oxygen and hydrolysis. Finally, chelating agents, such as disodium ethylenediaminetetraacetic acid, tetrasodium ethylenediaminetetraacetic acid, pentasodium ethylenediamine tetramethylene phosphonate, hydroxamic acids, citric acid, perform by binding metal ions thereby preventing their participation in oxidation reactions. Though chelating agents are relatively more stable compounds, they function by forming complexes with intrinsic oxidation catalysts (metal ions) instead of instead of restraining free radical propagation. Consequently, they may be less effective at reducing peroxidation.

Regarding the mechanisms to inhibit lipid peroxidation, each approach previously described has its advantages and disadvantages and they might be combined in different ways. For example, U.S. Pat. No. 7,012,092 claims a synergistic composition of tocopherols and tocotrienols designed to deliver at least 17-times the antioxidant capacity of pure synthetic tocopherol. However, Hanson, Gratton and Bardeen (2006) describe a combination of sunscreens that, after 60 minutes under UV radiation, exposure actually develops an antagonistic effect, increasing the production of ROS (Hanson, K. M., E. Gratton and C. J. Bardeen, “Sunscreen enhancement of UV-induced reactive oxygen species in the skin”, Free Rad. Bio. Med., 41, pp. 1205-1212 (2006).)

In view of this, there is a need in the art for new compounds or combination of compounds capable of providing effective protection against the noxious effects of UV radiation, whether on the skin or in personal care and pharmaceutical formulations.

### BRIEF SUMMARY OF THE INVENTION

The invention includes composition comprising a synergistically effective ratio of an amount of an hydroxamic acid, salt and/or a complex thereof and of an amount of an antioxidant. The composition may be capable of the reduction or prevention of lipid peroxidation upon exposure of a lipid to UV light. In some embodiments, the composition contains the hydroxamic acid, salt and/or a complex thereof and the antioxidant in a weight percent ratio of selected from those of about 5:about 12, about 4:about 11, about 3:about 10, about 1:about 4, about 1:about 6, about 1:about 15, about 15:about 1, about 6:about 1, about 4:about 1, about 10:about 3, about 11:about 4, about 12:about 5.

Also included are personal care formulations that exhibit reduced peroxidation of constituent lipids over a period of time wand which contain a synergistically effective ratio of an amount of at least one hydroxamic acid, salt and/or a complex thereof and of an amount of at least one antioxidant for use in the reduction of lipid peroxidation upon exposure of a lipid to UV light. The scope of the invention encompasses various methods including methods of increasing the antioxidant potential of an antioxidant, of reducing a rate of peroxidation of constituent lipids in a personal care formulation over a time period and of reducing peroxidation of lipids within mammalian skin cells, all including use of the disclosed compositions or formulations.

### BRIEF DESCRIPTION OF THE SEVERAL VIEWS OF THE DRAWINGS

The foregoing summary, as well as the following detailed description of embodiments of the invention, may be better understood when read in conjunction with the appended drawings. It should be understood, however, that the invention is not limited to the precise arrangements and instrumentalities shown. Moreover, at least one drawing executed in color is included herein. Copies of this patent or patent application publication with color drawings will be provided by the Office upon request and payment of the necessary fee.

In the drawings:

FIG. 1 shows plates before (A) and after (B) UVB irradiation, with highlighting of the samples showing synergistic effect;

FIG. 2 shows the data obtained from the plates A and B of FIG. 1, as a percent of control and the synergy index of the test composition as calculated by the Kull equation;

FIG. 3 shows the data obtained from Example 2, as a percent of control and the synergy index of the test composition as calculated by the Kull equation;

FIG. 4 shows the data obtained from Example 4 as a percent of control and the synergy index of the test composition as calculated by the Kull equation; and

FIG. 5 shows the data obtained from Example 5 as a percent of control and the synergy index of the test composition as calculated by the Kull equation.

### DETAILED DESCRIPTION OF THE INVENTION

The peroxidation of lipids and generation of free lipid radicals as a consequence of exposure of the lipids to UV light can result in damage to vital intracellular components or the cell membrane, including initiation of mutagenic activity, or degradation of the formulation in which the lipid was constituent. The invention described herein offers a remedy—the inventions described herein relate to the ability to prevent or reduce lipid peroxidation resulting from UV exposures. Specifically, compositions of an antioxidant agent and a chelating agent have been surprisingly demonstrated to act synergistically as evaluated using the Kull equation to increase lipid protection capacity by at least 50% in comparison to the use of each agent separately. This level of synergy has not been previously achieved and it is widely promising for industrial, medical and food applications in many different personal care and pharmaceutical products.

Thus, the invention described herein includes compositions that are capable of the reduction or prevention of lipid peroxidation upon exposure of a lipid to UV light, methods of increasing the antioxidant potential of an antioxidant, methods of reducing a rate of peroxidation of constituent lipids in a personal care formulation over a time period and methods of reducing peroxidation of lipids within a eukaryotic cell, preferably a skin cell, preferably a skin cell of a mammal. Formulations directed to pharmaceutical, personal care and/or food products that include the compositions of the invention are also included.

The invention described herein is capable of reducing lipid peroxidation of a lipid upon exposure to UVA, UVB, or UVC radiation, or a combination of two or more of the same (“UV light”).

“Lipid peroxidation”, as used herein, means the oxidative degradation of lipids by mechanisms that result in the removal of electrons from the lipid molecule to generate lipid radicals. The lipid(s) may be lipids within a cell (e.g., in situ intracellular lipids or membrane lipids in a living eukaryotic cell), lipids present in a food stuff (naturally-occurring or in an additive), or lipids of any origin outside a cell, such as the phospholipids found in lecithin, regardless of whether they are “neat” or incorporated into a formulation (“constituent lipids” of the formulation).

The occurrence/non-occurrence of lipid peroxidation may be quantified using any analytical technique known or to be developed in the art. Suitable techniques that include those that measure directly or indirectly the lipid peroxidation products generated. A suitable technique is disclosed in, for example, Pelle et al. from Estée Lauder (Pelle, E., D., Maes, G. A. Padulo, E. K. Kim and W. P. Smith, “An in vitro model to test relative antioxidant potential: ultraviolet-induced lipid peroxidation in liposomes”, Arch. Biochem. Biophys., 283, pp. 234-240 (1990)) as modified by Wang et al. (Wang, Y. R., H. Zhao, X. S. Sheng, P. E. Gambino, B. Costello and Bojanowski K., “Protective effect of Fructus lycii Polysaccharides against time- and hyperthermia-induced damage in cultured seminiferous epithelium”, J. Ethnopharmacol., 82, pp. 169-175 (2002)).

The inventive compositions and methods described herein may be used for cosmetic (aesthetic) and/or medical, including providing an anti-mutagenic effect when applied to living cells, particularly eukaryotic cells, such as skin cells, and/or for preservative, stability enhancing effects, when incorporated into personal care formulations, pharmaceutical formulations and/or food products.

Included in the invention is a mixture of two components, an antioxidant agent and a chelating agent that act synergistically when present together in an identified ratio. Specifically, the chelating agent is a hydroxamic acid or mixture of two or more hydroxamic acids. For use in the invention, the hydroxamic acid may include an alkylhydroxamic acid. The hydroxamic acids may be present in their free (un-neutralized) or salt (neutralized) forms, as well as their salts and/or complexes (or combinations of the same). Also included are those materials which are precursors to such compounds, salts and complexes, which, upon addition, react to form such compounds, salts and complexes.

As noted above, the hydroxamic acid(s) may be an alkylhydroxamic acid(s). Suitable alkylhydroxamic acid(s) may have linear or branched carbon chains of about two to about twenty-two carbon atoms, and preferably from about six to about twelve carbon atoms. The carbon chains may include double bonds, i.e., areas of unsaturation, and may also have functionality (by substitution of functional groups for the hydrogen atoms along the carbon chain, depending on desired end use and properties.)

For example, hydroxy groups may be beneficial side- or terminal-substituents on a chain of the selected hydroxamic acid, leading to better water compatibility. Other similar functional groups that meet the criteria of being compatible with and/or suggested for use in personal care and/or pharmaceutical formulations may be substituted for one of more hydrogen atom. Examples of compounds where such substitutions have been made include, without limitation, hexanohydroxamic acid, caprylohydroxamic acid, caprohydroxamic acid, laurohydroxamic acid.

In some embodiments, the hydroxamic acid, substituted or unsubstituted, selected for use in the invention may be synthesized from one or more natural oils using, e.g., lipase catalysis, as well as other hydroxamic synthesis techniques known or to be developed in the art.

In some embodiments, it is preferred that the hydroxamic acid, salt or complex as described above is present in an amount of about 0.001% to about 50% about 0.10% to 45%, about 5% to about 35% or about 10% to about 25%, each by weight of the total composition. If precursor materials are used as components according to one aspect of the invention, the weight percentages shall refer to the final amount of formed compound desired in the compositions.

The methods and compositions of the invention include an amount of an antioxidant. Any antioxidant or combination of antioxidants that is suitable for personal care, pharmaceutical or human or animal consumption may be used. Suitable examples may include, without limitation, ascorbic acid, sodium ascorbyl phosphate, ascorbyl palmitate, tocopherols, tocopheryl acetate, butylated hydroxytoluene, butylated hydroxyanisole, resveratrol, quercetin, uric acid, carotenes, glutathione, melatonin, and selenium. Depending on the contemplated end use, preferred antioxidants may include ascorbic acid, sodium ascorbyl phosphate, tocopherols, tocopheryl acetate and butylated hydroxytoluene or mixtures of two or more of these. In some embodiments, it is preferable that the selected antioxidant(s) is also a vitamin, so it may play a dual role in the formulation.

In some embodiments, it may be preferred that the antioxidant is present in an amount of about 0.001% to about 50.0%, about 5.0% to about 40.0%, about 15.0% to about 35%, and about 20.0% to about 28.0%, all of the total weight of the composition.

Notwithstanding the proposed amounts by weight of the total composition set out above, the hydroxamic acid, salt and/or a complex thereof and the antioxidant are present in the composition in a synergistically effective ratio relative to one another. As used herein, the term “synergistically effective ratio” means the presence of each of the agents in an amount relative to the other (a ratio) to provide a reduction or prevention of lipid peroxidation that is greater than the reduction/prevention of lipid peroxidation that results from either of the agents alone.

Such synergistic effect may be measured or evaluated by any means known or to be developed in the art. However, any effects deemed synergistic herein have been evaluated using the Kull equation. See, F. C. Kull et al.; Applied Microbiology Vol. 9, pp. 538-541 (1961); David C. Steinberg, Cosmetics & Toiletries Vol. 115 (No. 110), pp. 59-62, November 2000. Synergy between two compounds, or combination of compounds, is understood as the peculiar capacity of those compounds, or combination of compounds, to enhance or add to each others intrinsic activity when evaluated simultaneously (Synergy index <1.0). When the mixture of two different compounds or two different combinations of compounds results in no progress in activity, no synergy is observed (Synergy index=1.0). On the other hand, when the same mixture results in a decrease or inhibition of the intrinsic activity, the mixture is characterized as antagonist (Synergy index >1.0).

The Kull equation is as follows:

SI=C×D/A+C×E/B, where
“A” represents the response for substance A, or composition of substances A, at time “t”.
“B” represents the response for substance B, or composition of substances B, at time “t”.
“C” represents the Response for mixture A+B at time “t”.
“D” represents the amount of A in C, and
“E” represents the amount of B in C.

By “amount of” it is meant the amount used in the test when used alone (amount used in A or B) divided by the amount used in combination (amount used in C).

In some embodiments, it may be desirable that the synergistically effective ratio of hydroxamic acid to antioxidant is a ratio of the weight percent of hydroxamic acid, salt and/or a complex thereof to the weight percent of antioxidant (by weight of the total composition) chosen from the ratios of about 5:about 12, about 4:about 11, about 3:about 10, about 1:about 4, about 1:about 6, and about 1:about 15. In some embodiments, the ratios may be selected from about 15:about 1, about 6:about 1, about 4:about 1, about 10:about 3, about 11:about 4, about 12:about 5.

In some embodiments of the invention, the mixture of the hydroxamic acid, salt and/or a complex thereof and the antioxidant is carried in delivery vehicle which allows application of the mixture topically to a skin or other surface directly and/or which permits easy delivery of an amount of the composition to a secondary formulation, for example, a personal care formulation or a pharmaceutical formulation. Examples of the suitable vehicles may include, without limitation, water, distilled or purified water, alcohols, diols, glycols, esters, polyesters, vegetable oils, mineral oils, silicone oils, triglycerides, dimethyl isosorbide, waxes, talc, cornstarch, silica, chitosan, cellulose, tapioca, salts, and combinations so the same. In some embodiments, it may be preferred that the vehicle is a solution of water and glycols or dimethyl isosorbide.

Depending on the solubility and other physical-chemical properties of the antioxidants and of the alkylhydroxamic acids in the compositions of the invention, a solubilizing agent and/or an emulsifier may be required in the compositions herein described. Examples of appropriate solubilizing agents and/or emulsifiers include, but are not limited to glycerin, propylene glycol, butylene glycol, hexylene glycol, caprylyl glycol, ethylhexylglycerin, methylpropanediol, propanediol, 1,2-hexanediol, glyceryl caprylate, glyceryl undecylenate, propylene glycol dibenzoate, neopentyl glycol diheptanoate, ethanol, phenoxyethanol, phenethyl alcohol, benzyl alcohol, 2-(2-ethoxyethoxy)ethanol, polysorbate-20, polysorbate-60, polysorbate-80, alkyl glucosides, cocamides, and dimethyl isosorbide.

Preferably, the solubilizing agents and/or emulsifiers are present in an amount of 0.001% to 50.000%, or more preferably of 0.01% to 20.00%, by weight of the total composition of the invention.

Depending on the solubility and other physical-chemical properties of the selected antioxidant(s) and/or of the hydroxamic or alkylhydroxamic acids, inclusion of a solubilizing agent and/or an emulsifier in the compositions may be desirable. Suitable solubilizing agents and/or emulsifiers may include, but are not limited to glycerin, propylene glycol, butylene glycol, hexylene glycol, caprylyl glycol, ethylhexylglycerin, methylpropanediol, propanediol, 1,2-hexanediol, glyceryl caprylate, glyceryl undecylenate, propylene glycol dibenzoate, neopentyl glycol diheptanoate, ethanol, phenoxyethanol, phenethyl alcohol, benzyl alcohol, 2-(2-ethoxyethoxy)ethanol, polysorbate-20, polysorbate-60, polysorbate-80, alkyl glucosides, cocamides, and dimethyl isosorbide.

The compositions of the invention may be applied topically to a skin surface, preferable of a mammal (or otherwise introduced directly into a mammalian or eukaryotic cell), applied to a food surface or incorporated into a foodstuff to prevent or reduce lipid peroxidation upon exposure to UV light. Alternatively, the compositions of the invention may be incorporated into secondary formulations, such as personal care (including cosmetic and toiletry) formulations, and medicinal, pharmaceutical and/or food preservative formulations.

Such formulations include component other than the composition of the inventions. Components that may be present in the formulations will vary depending on the end use of the formulation but may include colorants, fragrances, active ingredients, sensates, pigments, surfactants, silicas, chitosan, alcohols, silicones, botanical extracts, gums, petrolatum, waxes, esters, texturizers, UV filtering agents, UV blocking agents, preservatives, vitamins, starch, and glycerin.

The formulations may be prepared to be any desirable product, for example, skin toners, skin cleansers, night creams, skin creams, shaving creams, skin care lotions, antiaging skin products, or cosmetic preparations; make-up, such as foundation, liquid and powder-based make-up, mascara, lipstick, blush, gloss, eye-liner and the like; or other personal care and/or pharmaceutical compositions, such as, ointments, gel-creams, lotions, sunscreens, lip balms, fragrances, massage oil, shampoos, conditioners, conditioning shampoos, hair styling gels, hair reparative, hair tonics, hair fixatives, hair mousses, bath and shower gels, liquid soaps, moisturizing sprays, makeup, pressed powder formulations, bath additives, ophthalmic preparations, foaming soaps and body washes, sunscreens, sanitizing wipes, hand sanitizers, towelettes and wipes and others.

If the composition of the invention is to be incorporated into a water-based secondary formulation, it may be preferred to prepare the composition as an hydrophilic dilution and to add it to the water phase of the secondary formulation.

With respect to an oil- or silicone-based formulation, one may prefer to prepare the compositions as an hydrophobic dilution and to add it to the oil or silicone phase of the formulation. In solid formulations, the compositions may be adsorbed onto a powdery matrix, such as, but not limited to silica, talc, magnesium carbonate, magnesium aluminum silicate, kaolin, titanium dioxide, zinc oxide, starch, modified starch, etc. Alternatively, the compositions are prepared as an emulsified system, or as a hydrophilic system, or a hydrophobic system, and added in the final step of the manufacturing process, preferably between 35-45° C. Such formulations can be prepared using operations known in the art or to be developed.

The amount of the composition present in any of the secondary formulations will necessarily vary depending on the end use of the formulation. However, it some embodiments one may prefer to include the composition in amounts of about 0.01% to about 50%, about 0.50% to about 20%, or about 0.1% to about 5% by weight of the entire formulation. If the composition of the invention is added to a formulation it may be premised and added to the formulation. Alternatively, each component of the composition may be added to the formulation separately.

The invention also includes a method of increasing the antioxidant potential of an antioxidant by combining the at least one antioxidant with a synergistically effective amount of an hydroxamic acid, salt and/or a complex thereof to form a synergistic blend, wherein the antioxidant potential of the synergistic blend is greater than the antioxidant potential of the antioxidant alone. The resultant product can be used in food, pharmaceutical and consumer products applications as nutritional supplement or preservative.

Methods of reducing a rate of peroxidation of constituent lipids in a personal care formulation over a time period and of reducing peroxidation of lipids within mammalian skin cells are also included within the scope of the invention.

All permutations of the elements described above, including the presence or absence of an element, are contemplated in the embodiments of the compositions, formulations, and/or the methods of the invention.

### Example 1

Test composition 1 was prepared to contain caprylohydroxamic acid (0.15% w/w) diluted in dimethyl isosorbide (0.4% w/w) and water (qsp 100% w/w). Test composition 2 contained ascorbic acid (0.5% w/w) diluted in the same solvent as test composition 1. Finally, test composition 3 was prepared containing both caprylohydroxamic acid (0.15% w/w) and ascorbic acid (0.5% w/w) diluted as test compositions 1 and 2.

UV-induced lipid peroxidation was evaluated for each of the test compositions 1-3, using the method develop by Pelle et al. from Estee Lauder (Pelle, E., D., Maes, G. A. Padulo, E. K. Kim and W. P. Smith, “An in vitro model to test relative antioxidant potential: ultraviolet-induced lipid peroxidation in liposomes”, Arch. Biochem. Biophys., 283, pp. 234-240 (1990)) as modified by Wang et al. (Wang, Y. R., H. Zhao, X. S. Sheng, P. E. Gambino, B. Costello and Bojanowski K., “Protective effect of Fructus lycii Polysaccharides against time- and hyperthermia-induced damage in cultured seminiferous epithelium”, J. Ethnopharmacol., 82, pp. 169-175 (2002)).

Briefly, the test compositions were added to dispersions of lecithin (a natural phospholipid) and irradiated separately with UVA light (6 mW/cm2, no lid) and with UVB light (0.9 mW/cm2). After 2.5 h of irradiation, thiobarbituric acid was added and the concentration of thiobarbituric acid reactive substances (TBARS,) such as malonaldehyde (malonaldehyde is a breakdown product generated spontaneously from oxidized lipids,) was measured spectrophotometrically at 550 nm, using Molecular Devices microplate reader MAX190. Distilled water was used as a negative control.

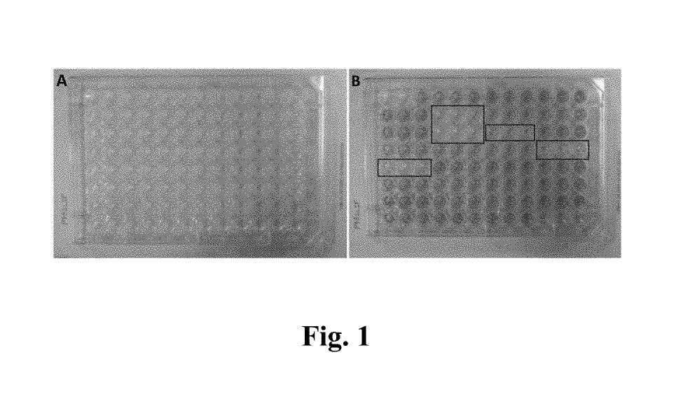

Samples were assayed in triplicates after dilution to the final concentration of 10%. Photographic documentation was taken with Canon Rebel X digital camera. Statistical significance was calculated with the double-tailed t-test and the p value significance threshold was set at 0.05 (differences with p values inferior to 0.05 were considered statistically significant). The results are reported as % CONTROL, which is defined as the ratio of TBARS formation relative to the water control. A positive control (ascorbic acid at 200 μg/mL) was included to provide technical validation to the experiment. FIG. 1 shows the photograph of plates before (A) and after (B) UVB irradiation, with highlights for synergistic samples.

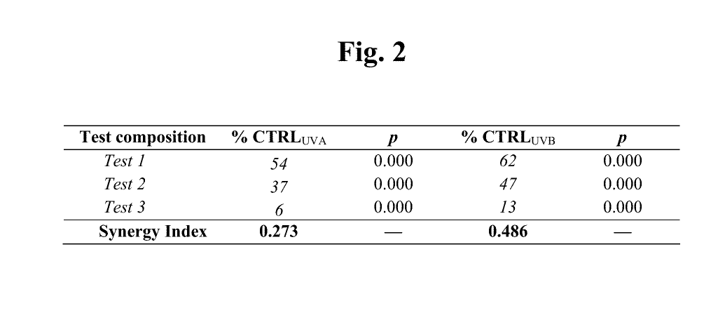

Suing the data, the synergy index for test composition 3 was calculated using the Kull equation. FIG. 2 presents the synergy index results for UV-induced lipid peroxidation.

### Example 2

In this example, test composition 4 was prepared to contain caprylohydroxamic acid (0.15% w/w) diluted in dimethyl isosorbide (0.4% w/w) and water (qsp 100% w/w). Test composition 5 contained a pool of antioxidants (1.8% w/w), which comprised ascorbic acid (0.4% w/w); sodium ascorbyl phosphate (0.4% w/w); α-tocopherol (0.4% w/w); tocopheryl acetate (0.4% w/w) and butylated hydroxytoluene (0.2% w/w) diluted in a mixture of dimethyl isosorbide (0.4% w/w), polysorbate-60 (0.3% w/w) and water (qsp 100% w/w). Finally, test composition 6 was prepared to contain both caprylohydroxamic acid (0.15% w/w) and the pool of antioxidants (1.8% w/w) diluted as test composition 2 was.

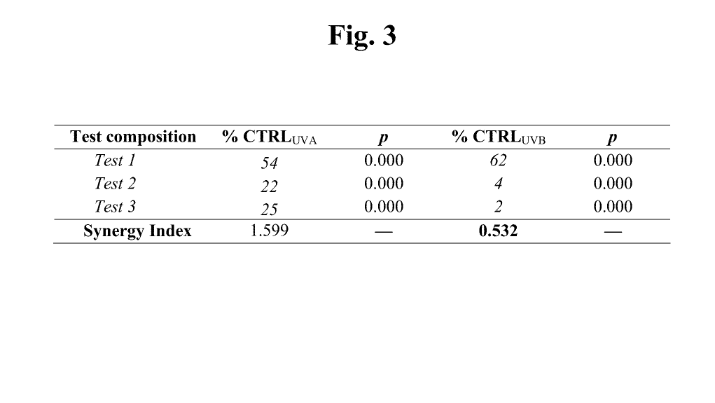

Evaluation of lipid peroxidation was carried out as in Example 1. Using the data generated, the synergy index for test composition 6 was calculated using the Kull equation. FIG. 3 presents the synergy index results for UV-induced lipid peroxidation.

### Example 3

A test composition 7 is prepared to contain caprylohydroxamic acid (0.15% w/w) diluted in dimethyl isosorbide (qsp 100% w/w). A test composition 8 is prepared to contain a combination of beta-carotene (0.5% w/w) and carrot oil (0.5% w/w) diluted in the same solvent as test composition 7. Finally, a test composition 9 is prepared to contain both caprylohydroxamic acid (0.15% w/w) and the combination of beta-carotene and carrot oil (0.5% w/w, respectively), diluted as test composition 7 is.

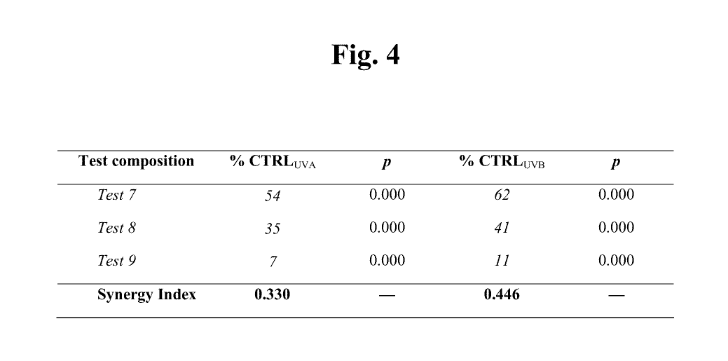

Evaluation of lipid peroxidation is carried out as in Example 1. Using the data generated, the synergy index for test composition 9 is calculated using the Kull equation. FIG. 4 presents the synergy index results for UV-induced lipid peroxidation.

### Example 4

A test composition 10 is prepared to contain caprylohydroxamic acid (0.15% w/w) diluted in dimethyl isosorbide (0.4% w/w) and water (qsp 100% w/w). A test composition 11 is prepared to contain both a hydroglycerin extract of Olea europaea leaves (2.0% w/w) and a hydroglycerin extract of Ficus carica fruit (2.0% w/w), available from ieS Labo in France, diluted in the same solvent as test composition 7. Finally, a test composition 12 is prepared to contain both caprylohydroxamic acid (0.15% w/w) and the combination of plant extracts (4.0% w/w) diluted as test composition 10 is.

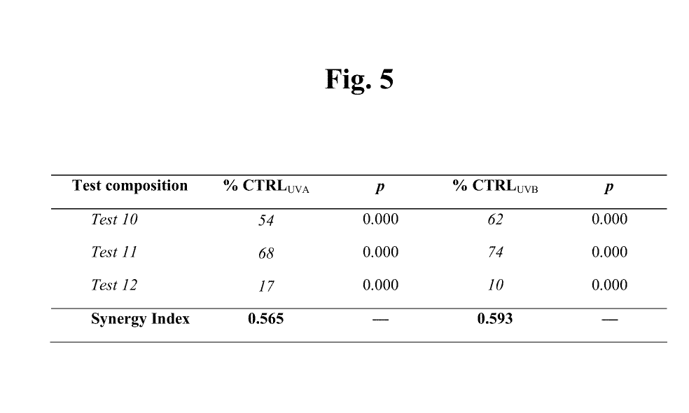

Evaluation of lipid peroxidation was carried out as in Example 1. Using the data generated, the synergy index for test composition 12 is calculated using the Kull equation. FIG. 5 presents the synergy index results for UV-induced lipid peroxidation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

Amendment to and listing of the claims:

1. (canceled)

2. A composition comprising a synergistically effective ratio of an amount of an hydroxamic acid, salt and/or a complex thereof and of an amount of an antioxidant wherein the composition is capable of the reduction or prevention of lipid peroxidation upon exposure of a lipid to UV light.

3. (canceled)

4. The composition of claim 2 wherein the UV light is selected from UVA, UVB and combinations thereof.

5. The composition of claim 2 wherein the hydroxamic acid, salt and/or a complex thereof is an alkylhydroxamic acid, salt and/or a complex thereof.

6. The composition of claim 2 wherein the hydroxamic acid, salt and/or a complex thereof has a carbon chain selected from a chain of 2 to 22 carbon atoms and a chain of 6 to 12 carbon atoms.

7. The composition of claim 2 wherein the hydroxamic acid, salt and/or a complex thereof has a carbon chain that is branched.

8. The composition of claim 2 wherein the hydroxamic acid, salt and/or a complex thereof has a carbon chain that is linear.

9. The composition of claim 2 wherein the hydroxamic acid, salt and/or a complex thereof has a carbon chain that contains at least one area of unsaturation.

10. The composition of claim 2 wherein the hydroxamic acid, salt and/or a complex thereof has a carbon chain that contains at least one area of substitution.

11. (canceled)

12. (canceled)

13. The composition of claim 2 wherein the hydroxamic acid, salt and/or a complex thereof is selected from hexanohydroxamic acid, caprylohydroxamic acid, caprohydroxamic acid, laurohydroxamic acid and combinations thereof.

14. The composition of claim 2 wherein the antioxidant is selected from ascorbic acid, sodium ascorbyl phosphate, ascorbyl palmitate, tocopherols, tocopheryl acetate, butylated hydroxytoluene, butylated hydroxyanisole, resveratrol, quercetin, uric acid, carotenes, glutathione, melatonin, selenium and mixtures thereof.

15. An antiaging composition for application to human skin comprising a synergistically effective ratio of an amount of at least one hydroxamic acid, salt and/or a complex thereof and of an amount of at least one antioxidant for use in the reduction of lipid peroxidation upon exposure of a lipid to UV light.

16. A composition comprising a synergistically effective ratio of an amount of at least one hydroxamic acid, salt and/or a complex thereof and of an amount of at least one antioxidant for use in the reduction of lipid peroxidation upon exposure of a constituent lipid in a personal care formulation to UV light.

17. A personal care formulation exhibiting reduced peroxidation of constituent lipids over a period of time comprising a synergistically effective ratio of an amount of at least one hydroxamic acid, salt and/or a complex thereof and of an amount of at least one antioxidant for use in the reduction of lipid peroxidation upon exposure of a lipid to UV light.

18. The formulation of claim 17 wherein the period of time is about 1 week to about 1 year.

19. The formulation of claim 17 wherein the UV light is selected from UVA, UVB and combinations thereof.

20. The formulation of claim 17 wherein the hydroxamic acid, salt and/or a complex thereof is an alkylhydroxamic acid, salt and/or a complex thereof.

21.-26. (canceled)

27. The formulation of claim 17 wherein the hydroxamic acid, salt and/or a complex thereof is synthesized from a natural oil.

28. The formulation of claim 17 wherein the hydroxamic acid, salt and/or a complex thereof is selected from hexanohydroxamic acid, caprylohydroxamic acid, caprohydroxamic acid, laurohydroxamic acid and combinations thereof.

29. The formulation of claim 17 wherein the antioxidant is selected from ascorbic acid, sodium ascorbyl phosphate, ascorbyl palmitate, tocopherols, tocopheryl acetate, butylated hydroxytoluene, butylated hydroxyanisole, resveratrol, quercetin, uric acid, carotenes, glutathione, melatonin, selenium and mixtures thereof.

30. (canceled)

31. The formulation of claim 17 further comprising a colorant, a fragrance, and a skin active ingredient.

32. The formulation of claim 17 in the form of a skin moisturizer, a skin toner, a skin cleanser, a night cream, a shaving cream or lotion, a make up foundation, a powder-based make-up, mascara, lipstick, blush, gloss, eye-liner, an ointments, a gel, a sunscreen, a lip balm, a massage oil, a shampoo, a hair conditioner, a hair styling composition, a bath and shower gel, a liquid soap, an ophthalmic preparation, and a wipe.

33. A method of increasing the antioxidant potential of an antioxidant comprising combining the at least one antioxidant with a synergistically effective amount of an hydroxamic acid, salt and/or a complex thereof to form a synergistic blend, wherein the antioxidant potential of the synergistic blend is greater than the antioxidant potential of the antioxidant alone.

34. A method of reducing a rate of peroxidation of constituent lipids in a personal care formulation over a time period comprising incorporating into the personal care formulation an effective amount of the composition of claim 2.
